(19) United States
(12) Patent Application Publication
(10) Pub. No.: US 2023/0121318 A1
(43) Pub. Date: Apr. 20, 2023

(54) REFRIGERATOR APPLIANCE WITH A MEAT AGING COMPARTMENT

(71) Applicant: Haier US Appliance Solutions, Inc., Wilmington, DE (US)

(72) Inventors: Srikanth Raavi Venkata, Louisville, KY (US); Abdel Hamad, Louisville, KY (US); Kyle James Tear, Louisville, KY (US)

(21) Appl. No.: 17/505,922

(22) Filed: Oct. 20, 2021

(51) Int. Cl. A23B 4/03; F25D 17/04; A61L 9/20

(52) U.S. Cl. CPC A23B 4/031; F25D 17/042; A61L 9/20; F25D 2317/04111; F25D 2400/361; F25D 2700/06; F25D 2700/02; A23V 2002/00; A61L 2209/11

(57) ABSTRACT

A refrigerator appliance includes a cabinet with a fresh food chamber, a freezer chamber, and an aging chamber each defined in the cabinet. The refrigerator appliance also includes an aging chamber door mounted to the cabinet and movable between a closed position and an open position. A method of operating the refrigerator appliance includes receiving a meat aging input from a user interface of the refrigerator appliance and moving a lock configured to retain the aging chamber door in the closed position to a locked position whereby the aging chamber door is retained in the closed position in response to the meat aging input. The method also includes activating a dedicated humidity control system in fluid communication with the aging chamber and fluidly isolated from the fresh food chamber and the freezer chamber for an aging time after moving the lock to the locked position.

### FIELD OF THE INVENTION

The present subject matter relates generally to refrigerator appliances, and more particularly to refrigerator appliances having convertible compartments.

### BACKGROUND OF THE INVENTION

Certain refrigerators appliances include multiple freezer compartments configured for maintaining different temperatures for storing different types of food and drink items. For example, a conventional quad door bottom mount refrigerator can include a freezer chamber having two separate freezer compartments that are maintained at different temperatures. More specifically, a first freezer compartment may be maintained at a conventional freezer temperature (e.g., around 0° F.), while a second “convertible” freezer compartment may be adjusted between a conventional freezer temperature and relatively warm temperatures.

Many consumers prefer meat that has been dry-aged. Dry-aging meat may reduce the moisture of the meat, and enhance the flavor. Dry-aging may also improve the texture of the meat due to enzymes breaking down the meat during the dry-aging process. However, dry-aging meat can be difficult in a home environment.

Accordingly, a refrigerator appliance including a convertible compartment with features for additional functions, such as dry-aging meat, would be useful.

### BRIEF DESCRIPTION OF THE INVENTION

Aspects and advantages of the invention will be set forth in part in the following description, or may be apparent from the description, or may be learned through practice of the invention.

In one exemplary aspect, a refrigerator appliance is provided. The refrigerator appliance includes a cabinet. A fresh food chamber, a freezer chamber, and an aging chamber are each defined in the cabinet. The refrigerator appliance also includes a dedicated humidity control system. The dedicated humidity control system is in fluid communication with the aging chamber and is fluidly isolated from the fresh food chamber and the freezer chamber. The refrigerator appliance further includes an aging chamber door mounted to the cabinet such that the aging chamber door is movable relative to the cabinet between a closed position where the aging chamber is sealingly enclosed by the aging chamber door and an open position permitting access to the aging chamber. The refrigerator appliance also includes a lock configured to retain the aging chamber door in the closed position and a controller. The controller is configured for receiving a meat aging input from a user interface of the refrigerator appliance and moving the lock to a locked position so that the aging chamber door is retained in the closed position in response to the meat aging input. The controller is further configured for activating the dedicated humidity control system for an aging time after moving the lock to the locked position.

In another exemplary aspect, a method of operating a refrigerator appliance is provided. The refrigerator appliance includes a cabinet with a fresh food chamber, a freezer chamber, and an aging chamber each defined in the cabinet. The refrigerator appliance further includes an aging chamber door mounted to the cabinet such that the aging chamber door is movable relative to the cabinet between a closed position where the aging chamber is sealingly enclosed by the aging chamber door and an open position permitting access to the aging chamber. The method includes receiving a meat aging input from a user interface of the refrigerator appliance. In response to the meat aging input, the method includes moving a lock configured to retain the aging chamber door in the closed position to a locked position whereby the aging chamber door is retained in the closed position. The method also includes activating a dedicated humidity control system in fluid communication with the aging chamber and fluidly isolated from the fresh food chamber and the freezer chamber for an aging time after moving the lock to the locked position.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a perspective view of a refrigerator appliance according to an exemplary embodiment of the present subject matter.

FIG. 2 provides a front view of the exemplary refrigerator appliance of FIG. 1 with the aging chamber door of the refrigerator appliance shown in an open position.

FIG. 3 provides an enlarged view of a portion of the exemplary refrigerator appliance of FIG. 1.

FIG. 4 illustrates an exemplary user interface on a remote user interface device according to one or more embodiments of the present disclosure which is operable with a refrigerator appliance such as the refrigerator appliance of FIG. 1.

FIG. 5 provides another view of the exemplary user interface of FIG. 4.

FIG. 6 provides a flow chart illustrating an exemplary method of operating a refrigerator appliance according to an exemplary embodiment of the present subject matter.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “first,” “second,” and “third” may be used interchangeably to distinguish one component from another and are not intended to signify location or importance of the individual components. The terms “upstream” and “downstream” refer to the relative direction with respect to fluid flow in a fluid pathway. For example, “upstream” refers to the direction from which the fluid flows, and “downstream” refers to the direction to which the fluid flows. Terms such as “inner” and “outer” refer to relative directions with respect to the interior and exterior of the refrigerator appliance, and in particular the food storage chamber(s) defined therein. For example, “inner” or “inward” refers to the direction towards the interior of the refrigerator appliance. Terms such as “left,” “right,” “front,” “back,” “top,” or “bottom” are used with reference to the perspective of a user accessing the refrigerator appliance. For example, a user stands in front of the refrigerator to open the doors and reaches into the food storage chamber(s) to access items therein.

As used herein, terms of approximation such as “generally,” “about,” or “approximately” include values within ten percent greater or less than the stated value. When used in the context of an angle or direction, such terms include within ten degrees greater or less than the stated angle or direction, e.g., “generally vertical” includes forming an angle of up to ten degrees in any direction, e.g., clockwise or counterclockwise, with the vertical direction V.

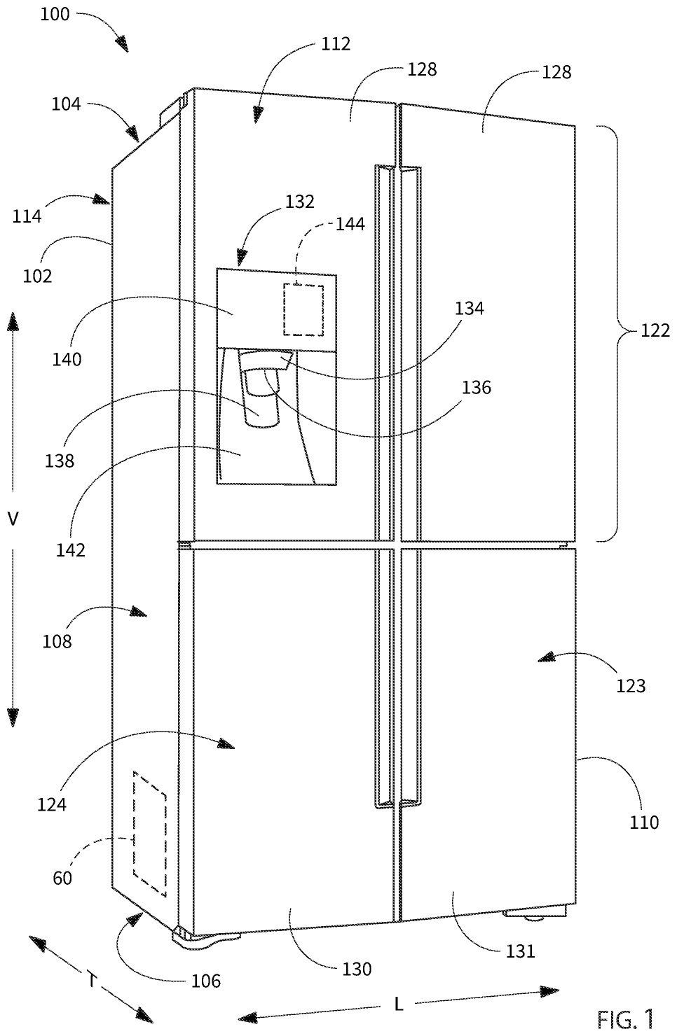

FIG. 1 provides a perspective view of a refrigerator appliance 100 according to an exemplary embodiment of the present subject matter. Refrigerator appliance 100 includes a housing or cabinet 102 that extends between a top 104 and a bottom 106 along a vertical direction V, between a left side 108 and a right side 110 along a lateral direction L, and between a front side 112 and a rear side 114 along a transverse direction T. Each of the vertical direction V, lateral direction L, and transverse direction T are mutually perpendicular to one another and form an orthogonal direction system.

Cabinet 102 defines chilled chambers for receipt of food items for storage. In particular, cabinet 102 defines fresh food chamber 122 positioned at or adjacent top 104 of cabinet 102 with a freezer chamber 124 and an aging chamber 123 arranged at or adjacent bottom 106 of cabinet 102. As such, refrigerator appliance 100 is generally referred to as a bottom mount refrigerator. It is recognized, however, that the benefits of the present disclosure apply to other types and styles of refrigerator appliances such as, e.g., a top mount refrigerator appliance or a side-by-side style refrigerator appliance. As another example, although the illustrated example embodiment depicts the freezer chamber 124 on the left side and the aging chamber 123 on the right side, it is recognized that such configuration is provided by way of example only and not limitation, e.g., the freezer chamber 124 and the aging chamber 123 may be transposed in some embodiments. Consequently, the description set forth herein is for illustrative purposes only and is not intended to be limiting in any aspect to any particular refrigerator chamber configuration.

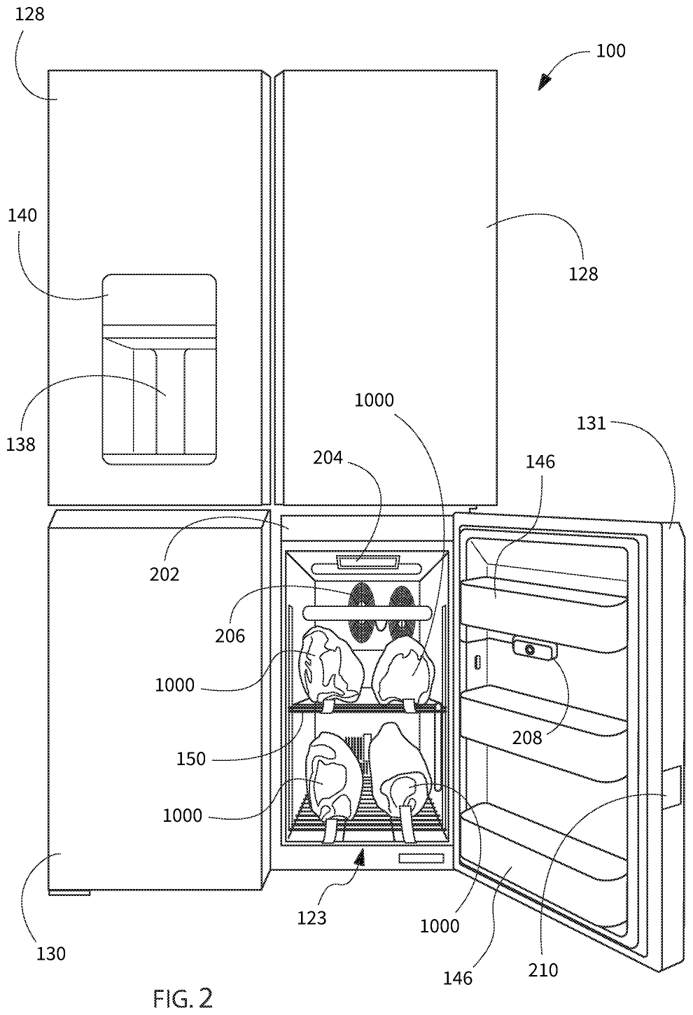

Refrigerator doors 128 are each rotatably hinged to a corresponding edge of cabinet 102 for selectively accessing fresh food chamber 122. Similarly, freezer door 130 and aging chamber door 131 are rotatably hinged to an edge of cabinet 102 in the illustrated example embodiment for selectively accessing freezer chamber 124 and aging chamber 123. As another example, one or both of the freezer door 130 and the aging chamber door 131 may instead be a front portion of a slidable drawer which can be selectively moved in and out of the respective chamber 123 and/or 124 along transverse direction T. To prevent leakage of cool air, the doors 128, 130, 131, and/or cabinet 102 may define one or more sealing mechanisms (e.g., rubber gaskets, not shown) at the interface where the doors 128, 130, 131 meet cabinet 102. Refrigerator doors 128, freezer door 130, and aging chamber door 131 are shown in the closed configuration in FIG. 1, and aging chamber door 131 is shown in the open configuration in FIG. 2. It should be appreciated that doors having a different style, position, or configuration are possible and within the scope of the present subject matter.

In an exemplary embodiment, cabinet 102 also defines a mechanical compartment 60 at or near the bottom 106 of the cabinet 102 for receipt of a hermetically sealed cooling system configured for transporting heat from the inside of the refrigerator to the outside. One or more ducts may extend between the mechanical compartment 60 and the chilled chambers 122, 123, and/or 124 to provide fluid communication therebetween, e.g., to provide chilled air from the hermetically sealed cooling system, e.g., from an evaporator thereof, to one or more of the chilled chambers 122, 123, and/or 124. As is generally understood by those of skill in the art, the hermetically sealed system contains a working fluid, e.g., refrigerant, which flows between various heat exchangers of the sealed system where the working fluid changes phases. For example, the hermetically sealed system includes at least one evaporator where the working fluid absorbs thermal energy and changes from a liquid state to a gas state and at least one condenser where the working fluid releases thermal energy and returns to the liquid state from the gas state. As is understood, because the system is sealed, the working fluid is contained within the system and travels between the heat exchangers of the hermetically sealed system. A fan is typically provided at each heat exchanger of the sealed system. For example, a fan may force air across and around the at least one evaporator to transfer thermal energy from the air to the evaporator (and more particularly, to the working fluid therein), thereby generating a flow of chilled air which may be provided to one or more of the chilled chambers 122, 123, and/or 124. In some embodiments, some components of the sealed system may be located on different sides of a thermally insulated barrier, e.g., the at least one condenser may be positioned outside of the thermally insulated barrier with respect to the chilled chambers such that heat released from the working fluid as it condenses is directed away from the chilled chambers and to an ambient environment around the refrigerator appliance 100, and the at least one evaporator may be positioned on the same side of the thermally insulated barrier as the chilled chambers, whereby the flow of chilled air from the evaporator(s) to the chilled chambers may be entirely contained within a thermally insulated enclosure.

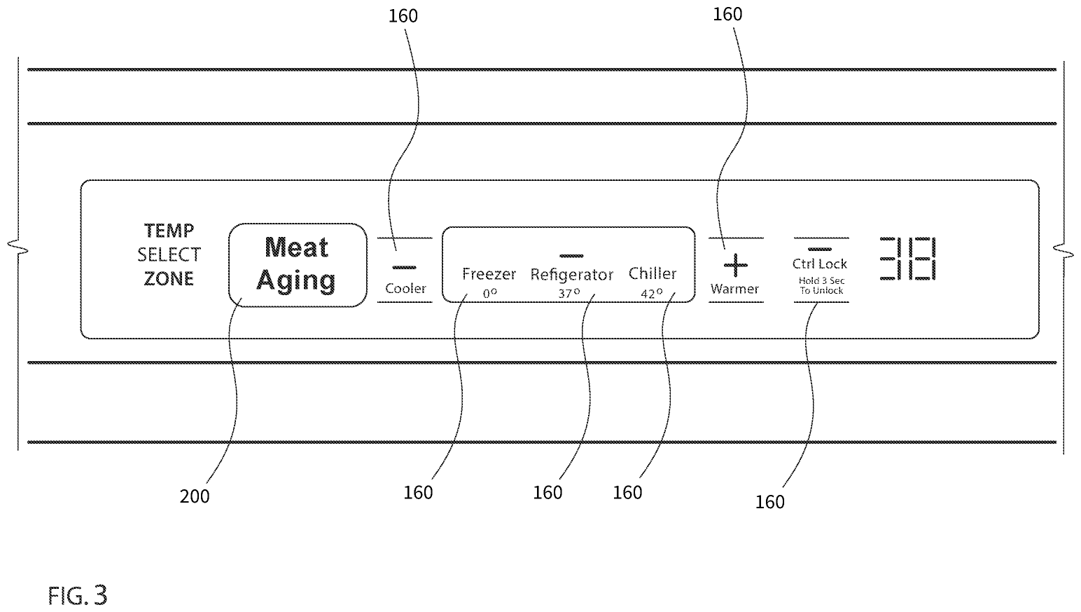

Refrigerator appliance 100 also includes a dispensing assembly 132 for dispensing liquid water and/or ice. Dispensing assembly 132 includes a dispenser 134 positioned on or mounted to an exterior portion of refrigerator appliance 100, e.g., on one of refrigerator doors 128. Dispenser 134 includes a discharging outlet 136 for accessing ice and liquid water. An actuating mechanism 138, shown as a paddle, is mounted below discharging outlet 136 for operating dispenser 134. In alternative exemplary embodiments, any suitable actuating mechanism may be used to operate dispenser 134. For example, dispenser 134 can include a sensor (such as an ultrasonic sensor) or a button rather than the paddle. A control panel 140 is provided for controlling the mode of operation. For example, control panel 140 includes a plurality of user inputs 160 (see, e.g., FIG. 3), such as a water dispensing button and an ice-dispensing button, for selecting a desired mode of operation such as crushed or non-crushed ice.

Discharging outlet 136 and actuating mechanism 138 are an external part of dispenser 134 and are mounted in a dispenser recess 142. Dispenser recess 142 is positioned at a predetermined elevation convenient for a user to access ice or water and enabling the user to access ice without the need to bend-over and without the need to open refrigerator doors 128. In the exemplary embodiment, dispenser recess 142 is positioned at a level that approximates the chest level of an adult user. According to an exemplary embodiment, the dispensing assembly 132 may receive ice from an icemaker disposed in a sub-compartment of the fresh food chamber 122.

Refrigerator appliance 100 further includes a controller 144. Operation of the refrigerator appliance 100 is regulated by controller 144 that is operatively coupled to control panel 140. In some exemplary embodiments, control panel 140 may represent a general purpose I/O (“GPIO”) device or functional block. In some exemplary embodiments, control panel 140 may include input components, such as one or more of a variety of electrical, mechanical or electro-mechanical input devices including rotary dials, push buttons, touch pads, and touch screens. Control panel 140 can be communicatively coupled with controller 144 via one or more signal lines or shared communication busses. Control panel 140 provides selections for user manipulation of the operation of refrigerator appliance 100, e.g., whereby a user may provide one or more set point temperatures for the various compartments 122, 123, and 124. In response to user manipulation of the control panel 140, controller 144 operates various components of refrigerator appliance 100. For example, controller 144 is operatively coupled or in communication with various airflow components, e.g., dampers and fans, as discussed below. Controller 144 may also be communicatively coupled with a variety of sensors, such as, for example, chamber temperature sensors or ambient temperature sensors. Such chamber temperature sensors and/or ambient temperature sensors may be or include thermistors, thermocouples, or any other suitable temperature sensor. Controller 144 may receive signals from these temperature sensors that correspond to the temperature of an atmosphere or air within their respective locations.

Controller 144 includes memory and one or more processing devices such as microprocessors, CPUs or the like, such as general or special purpose microprocessors operable to execute programming instructions or micro-control code associated with operation of refrigerator appliance 100. The memory can represent random access memory such as DRAM, or read only memory such as ROM or

FLASH. The processor executes programming instructions stored in the memory. The memory can be a separate component from the processor or can be included onboard within the processor. Alternatively, controller 144 may be constructed without using a microprocessor, e.g., using a combination of discrete analog and/or digital logic circuitry (such as switches, amplifiers, integrators, comparators, flip-flops, AND gates, and the like) to perform control functionality instead of relying upon software. The controller 144 may be positioned in a variety of locations throughout refrigerator appliance 100. In the illustrated embodiment, the controller 144 is located within a control panel area 140 of one of the refrigerator doors 128, as shown in FIG. 1. In other example embodiments, the controller 144 may be positioned at or near the rear side 114 and/or the bottom 106 of the refrigerator appliance 100.

Controller 144 may include, or be associated with, one or more memory elements or non-transitory computer-readable storage mediums, such as RAM, ROM, EEPROM, EPROM, flash memory devices, magnetic disks, or other suitable memory devices (including combinations thereof). These memory devices may be a separate component from the processor or may be included onboard within the processor. In addition, these memory devices can store information and/or data accessible by the one or more processors, including instructions that can be executed by the one or more processors. It should be appreciated that the instructions can be software written in any suitable programming language or can be implemented in hardware.

Additionally, or alternatively, the instructions can be executed logically and/or virtually using separate threads on one or more processors.

For example, controller 144 may be operable to execute programming instructions or micro-control code associated with an operating cycle of appliance 100. In this regard, the instructions may be software or any set of instructions that when executed by the processing device, cause the processing device to perform operations, such as running one or more software applications, displaying a user interface, receiving user input, processing user input, etc. Moreover, it should be noted that controller 144 as disclosed herein is capable of and may be operable to perform any methods, method steps, or portions of methods as disclosed herein. For example, in some embodiments, methods disclosed herein may be embodied in programming instructions stored in the memory and executed by controller 144.

FIG. 2 provides a front view of refrigerator appliance 100 with refrigerator doors 128 and freezer door 130 in the closed position while aging chamber door 131 is shown in an open position. As those of ordinary skill in the art will recognize, various storage components may be mounted within fresh food chamber 122 and freezer chamber 124 to facilitate storage of food items therein. Particular examples of such storage components are depicted within the aging chamber 123 in the example embodiment illustrated in FIG. 2, which exemplary storage components include bins 146 and shelves 150 in the aging chamber 123. In additional exemplary embodiments, such storage components may also or instead include drawers. Various combinations of any or all such exemplary storage components may be provided, e.g., mounted, within fresh food chamber 122, aging chamber 123, and/or freezer chamber 124.

As may be seen in FIG. 2, one or more exemplary pieces of meat 1000, e.g., beef, may be placed in the aging chamber 123. For example, the meat 1000 may be placed on shelves 150 within the aging chamber 123, and the shelves 150 may be formed of an open construction, such as wire shelves, in order to promote air flow through the shelves 150 and around each piece of meat 1000 on all sides.

Also as may be seen in FIG. 2, a humidity control system 202 may be provided in the aging chamber 123. The humidity control system 202 may be a dedicated humidity control system, e.g., dedicated solely to controlling humidity within the aging chamber 123. The aging chamber 123 and/or the humidity control system 202 may be fluidly isolated from the fresh food chamber 122 and the freezer chamber 124. For example, chilled air from the sealed system in the mechanical compartment 60 may be provided to the aging chamber 123 by a dedicated duct that is isolated from and not coupled to the fresh food chamber 122 or the freezer chamber 124.

In some embodiments, the humidity control system 202 may be a box or insert that is mounted inside the aging chamber 123. The humidity control system 202 may include a fan 206, a light 204 configured to emit ultraviolet light, such as a light-emitting diode (LED) that emits UV light, such as UV-C light. The humidity control system 202 may also include a dedicated controller (not labelled) in operative communication with the fan 206 and the light 204 such that the dedicated controller directs or commands operation of the components of the humidity control system 202, e.g., the light 204 and the fan 206 of the humidity control system 202. The controller of the humidity control system 202 may further be in communication with the main controller 144 of the refrigerator appliance 100, such as the controller of the humidity control system 202 may be a slave controller and the controller 144 may be a master controller.

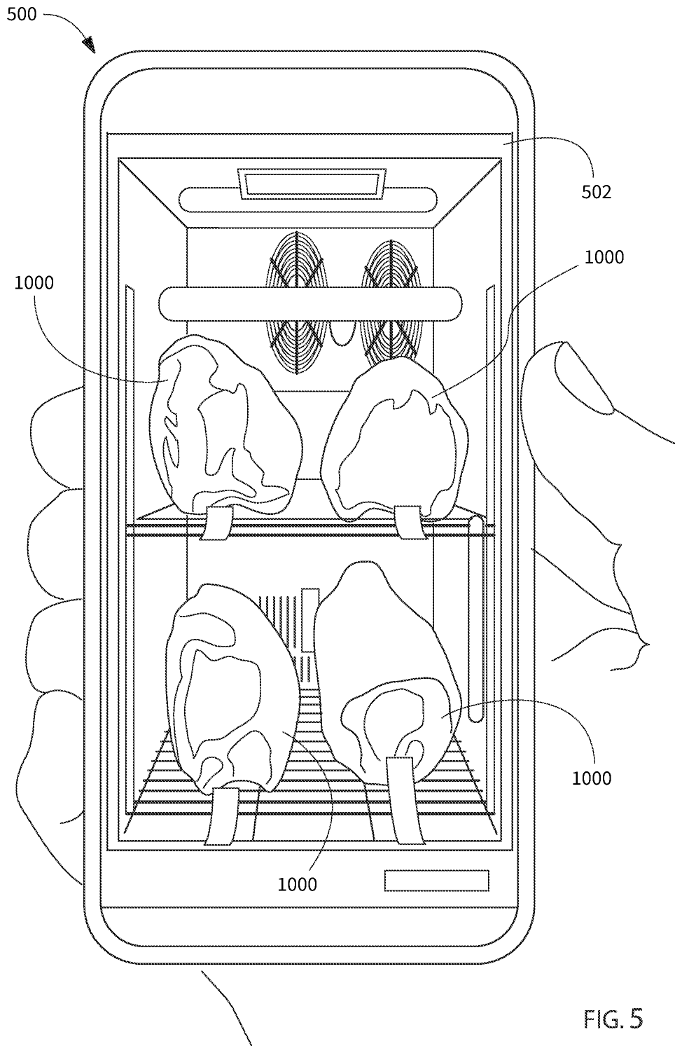

Also as may be seen in FIG. 2, the refrigerator appliance 100 may further include a camera 208. The camera 208 may be positioned within the refrigerator appliance 100, such as within the aging chamber 123, such that the aging chamber 123 is within a field of view of the camera 208. In particular, the camera 208 may be positioned such that the aging chamber 123, e.g., the shelves 150 in the aging chamber 123 and contents, e.g., meat 1000, that may be disposed thereon, is within the field of view at least when the aging chamber door 131 is in the closed position. For example, the camera 208 may be mounted on an interior surface of the door 131, as illustrated in FIG. 2. In additional embodiments, the camera 208 may be mounted within the cabinet 102 or in other suitable location such that the aging chamber 123 is within the field of view of the camera 208. In such embodiments, the controller 144 may be further configured for receiving an image from the camera 208 and transmitting the image to a display, which may be physically connected to the refrigerator appliance 100, such as on an exterior surface of one of the doors 128, such as incorporated in or adjoining the control panel 140, or which may be a display of a remote user interface device 500 (see, e.g., FIG. 5).

In some embodiments, the aging chamber door 131 may include a lock 210. The lock 210 may be configured to retain the aging chamber door 131 in the closed position, such as the lock 210 may be movable between an unlocked position which permits opening of the door 131 and a locked position wherein, when the lock 210 is moved to the locked position while the door 131 is in the closed position, the aging chamber door 131 is retained in the closed position by the lock 210. The lock 210 may engage the cabinet 102 to retain the door 131 in the closed position. For example, the lock 210 may be an electromagnetic lock. The lock 210 may be actuated by the controller 144, e.g., the controller 144 may be configured to move the lock 210 from the locked position to the unlocked position and vice versa.

Turning now to FIG. 3, a plurality of exemplary user inputs 160 of the refrigerator appliance 100 are illustrated. As mentioned above, the user inputs 160 may be positioned on the control panel 140. The user inputs 160 may also or instead be located on an external surface of one of the doors, such as on a top surface of the aging chamber door 131. In some embodiments, the refrigerator appliance 100 may also include a meat aging input selector 200. In such embodiments, pressing or otherwise actuating the meat aging input selector 200 may initiate a meat aging mode or meat aging operation in the aging chamber 123, as will be described in more detail below.

The chambers of the refrigerator appliance 100 are chilled chambers, e.g., each chamber 122, 124, and 123 may be operable at temperatures below room temperature, where room temperature is understood to range from about 66° F. to about 78° F. For example, the temperature of the fresh food chamber 122 may be within a range between approximately thirty-four degrees Fahrenheit (34° F.) and approximately forty-two degrees Fahrenheit (42° F.). Also by way of example, the temperature of the freezer chamber 124 may be within a range between approximately negative six degrees Fahrenheit (−6° F.) and approximately six degrees Fahrenheit (6° F.). It should be understood that fresh food chamber 122 and freezer chamber 124 may be selectively operable at any number of various temperatures and/or temperature ranges as desired or required per application. The aging chamber 123 may be usable as a convertible chamber for storing fresh or frozen items, e.g., the aging chamber may be operable within a range of temperatures between approximately negative six degrees Fahrenheit (−6° F.) and approximately forty-two degrees Fahrenheit (42° F.), including at particular temperatures within the foregoing range in a specific meat-aging operation, which will be described in more detail below.

As mentioned above, the aging chamber 123 may be operable within a range of temperatures between approximately negative six degrees Fahrenheit (−6° F.) and approximately forty-two degrees Fahrenheit (42° F.). The operating temperature of the aging chamber 123 may be determined by a temperature setting, e.g., a user-selected set point temperature, which may be received by the controller 144, such as from one of the inputs 160 illustrated in FIG. 3, as described above. In some embodiments, the operating temperature of the aging chamber 123 may be set to a predetermined temperature based on a selected operating mode. For example, the meat aging mode may include setting a temperature in the aging chamber to an aging temperature in response to the meat aging input. The aging temperature may be between about 35° F. and about 45° F., such as about 40° F. or about 37° F.

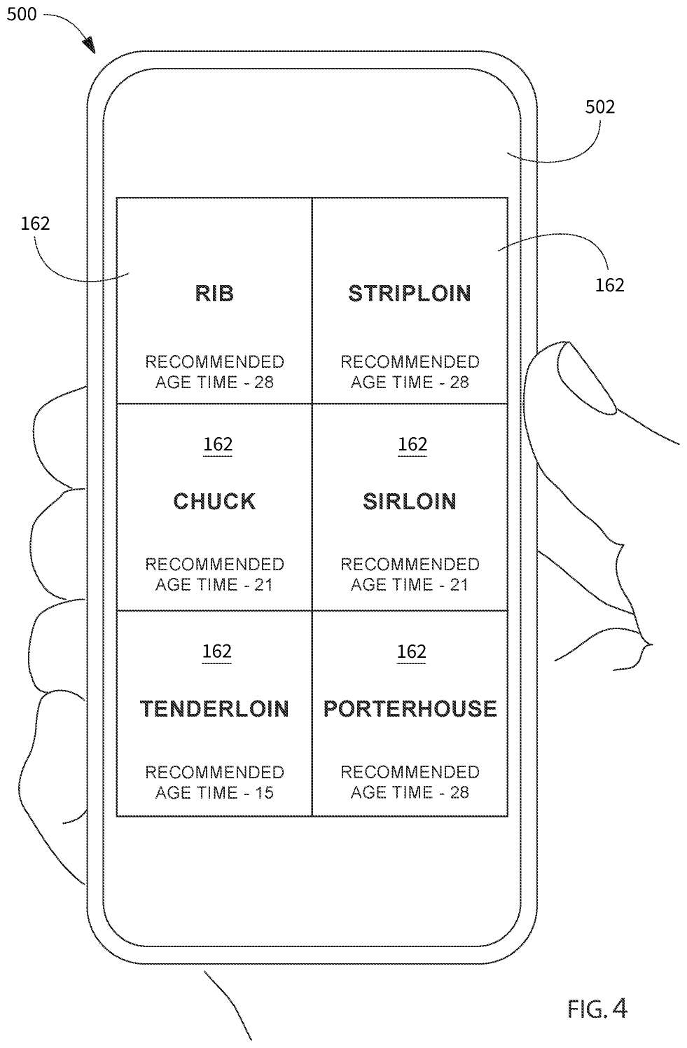

Turning now to FIGS. 4 and 5, in some embodiments, the meat aging operation may be performed in part by a remote user interface device 500. The remote user interface device 500 may include a display 502, such as a touchscreen display. The display 502 may be operable to provide instructions or information to a user. In embodiments where the display 502 is a touchscreen, the display 502 may also receive input from the user.

The refrigerator appliance 100 may be in communication with the remote user interface device 500 device through various possible communication connections and interfaces. The refrigerator appliance 100 and the remote user interface device 500 may be matched in wireless communication, e.g., connected to the same wireless network. The refrigerator appliance 100 may communicate with the remote user interface device 500 via short-range radio such as BLUETOOTH® or any other suitable wireless network having a layer protocol architecture. As used herein, “short-range” may include ranges less than about ten meters and up to about one hundred meters. For example, the wireless network may be adapted for short-wavelength ultra-high frequency (UHF) communications in a band between 2.4 GHz and 2.485 GHz (e.g., according to the IEEE 802.15.1 standard). In particular, BLUETOOTH® Low Energy, e.g., BLUETOOTH® Version 4.0 or higher, may advantageously provide short-range wireless communication between the refrigerator appliance 100 and the remote user interface device 500. For example, BLUETOOTH® Low Energy may advantageously minimize the power consumed by the exemplary methods and devices described herein due to the low power networking protocol of BLUETOOTH® Low Energy.

The remote user interface device 500 is “remote” at least in that it is spaced apart from and not physically connected to the refrigerator appliance 100, e.g., the remote user interface device 500 is a separate, stand-alone device from the refrigerator appliance 100 which communicates with the refrigerator appliance 100 wirelessly. Any suitable device separate from the refrigerator appliance 100 that is configured to provide and/or receive communications, information, data, or commands from a user may serve as the remote user interface device 500, such as a smartphone (e.g., as illustrated in FIGS. 4 and 5), smart watch, personal computer, smart home system, or other similar device. For example, the remote user interface device 500 may be a smartphone operable to store and run applications, also known as “apps,” and some or all of the method steps disclosed herein may be performed by a smartphone app.

The remote user interface device 500 may include a memory for storing and retrieving programming instructions. Thus, the remote user interface device 500 may provide a remote user interface which may be an additional user interface to the control panel 140. For example, the remote user interface device 500 may be a smartphone operable to store and run applications, also known as “apps,” and the remote user interface may be provided as a smartphone app.

Referring now specifically to FIG. 4, in some embodiments, the remote user interface device 500 may be operable to display a plurality of meat type selections 162, non-limiting examples of possible meat type selections are illustrated in FIG. 4. The user may then select a meat type, e.g., by pressing a portion of the touchscreen display 502 corresponding to the type of meat to be aged in the aging chamber 123, thereby generating a meat type input, which may be transmitted to the refrigerator appliance 100, such as to the controller 144 thereof. As will be described further below, an aging time of the dry-aging operation may be determined based at least in part on the meat type. In the example embodiment illustrated in FIG. 4, an exemplary recommended aging time (in days) for each meat type is included with the respective meat type selection 162.

Turning now to FIG. 5, the remote user interface device 500 may be operable to receive and display an image from the camera 208. For example, the image from the camera 208 may be displayed on the display 502 of the remote user interface device 500 in some embodiments. Thus, for example, a user may be able to view the pieces of meat 1000 as they are aging within the aging chamber 123, even while the door 131 is locked.

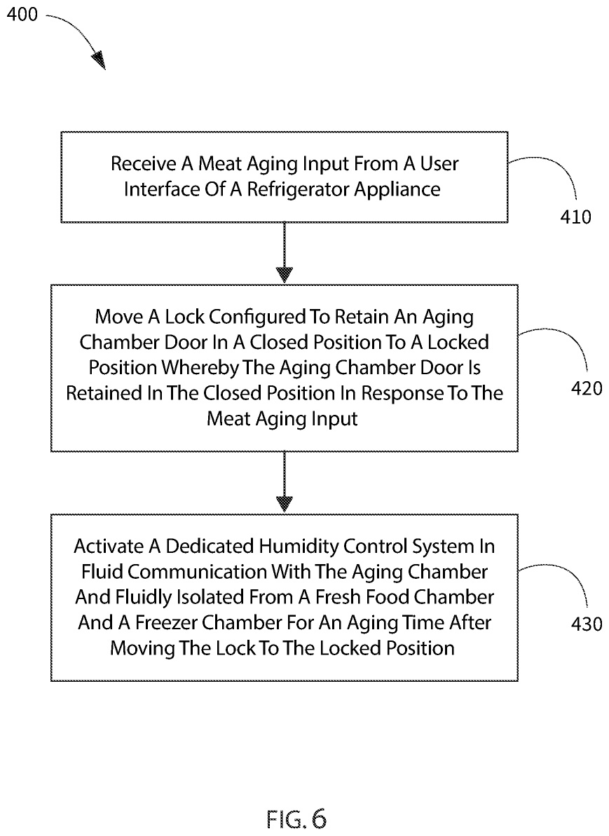

Embodiments of the present disclosure may include methods of operating a refrigerator appliance such as the exemplary method 400 illustrated in FIG. 6. For example, the controller 144 may be operable to perform some or all of the steps of the method 400. As mentioned, the method 400 may include a step 410 of receiving a meat aging input from a user interface of the refrigerator appliance. For example, the meat aging input may be received from a user interface on the refrigerator appliance, such as the meat aging input selector 200 illustrated in FIG. 3 and described above. In additional embodiments, the meat aging input may be received from a remote user interface, e.g., wirelessly communicating with the refrigerator appliance, such as on a remoter user interface device, e.g., as illustrated in FIGS. 4 and 5 and described above.

In response to the meat aging input, the method 400 may then proceed to a step 420 of locking the aging chamber door. For example, step 420 may include moving a lock configured to retain the aging chamber door in the closed position to a locked position, thus the aging chamber door is retained in the closed position by the lock. The dry-aging process is preferably carried out with controlled temperature, humidity, and air flow. Thus, locking the aging chamber door may prevent undesired fluctuations in, e.g., temperature and/or humidity within the aging chamber, such as due to ambient air (which is generally warmer and more humid than the air within the aging chamber) entering the chamber when the door is opened.

In order to promote the desired temperature and humidity for dry-aging, as mentioned above, the method 400 may further include a step 430 of activating a dedicated humidity control system for an aging time after moving the lock to the locked position. For example, the humidity control system may be dedicated to the aging chamber in that the humidity control system is in fluid communication with the aging chamber and fluidly isolated from other portions of the refrigerator appliance, such as the fresh food chamber and the freezer chamber.

The method 400 may, in some embodiments, further include providing one or more user notifications. For example, in some embodiments, the method 400 may include providing a user notification after the aging time has elapsed and/or when the aging time remaining is less than a threshold, such as less than 24 hours, or less than 8 hours, etc.

As mentioned above, the dedicated humidity control system may include one or both of a light configured to emit ultraviolet (UV) light and/or a fan. In some embodiments, the step 430 of activating the dedicated humidity control system may include activating the light periodically throughout the duration of the aging time. As a result of such activation, ultraviolet (UV) light may be directed into the aging chamber while the light is activated. The UV light, which may be, e.g., a UV-C LED, may be activated for various periods. For example, activating the light periodically may include activating the light for between about two minutes per hour and about ten minutes per hour, such as between about three minutes per hour and about eight minutes per hour, such as about five minutes per hour. Activating the UV light during the aging time generally sterilizes the air within the aging chamber, e.g., may disinfect the aging chamber, may remove or reduce fungus and other contaminants, and may reduce the odor accumulation from the aging meat during the aging time.

In additional embodiments, the step 430 of activating the dedicated humidity control system may also or instead include rotating the fan to motivate air within and throughout the aging chamber during the aging time. For example, step 430 may include continuously rotating the fan at a constant speed throughout the duration of the aging time, whereby air is motivated to circulate within the aging chamber for the entire aging time. The constant speed at which the fan is rotated may be a relatively low speed, to provide a relatively low air speed within the aging chamber and/or around meat 1000 therein. For example, the air speed within the aging chamber may be less than about 2 meters per second, such as less than about 1.5 meters per second, such as about 1 meter per second or less, such as about 1 meter per second, such as about 1.2 meters per second. Also by way of example, the air speed at the shelves 150 (and the meat 1000 thereon) may be less than about 2 meters per second, such as less than about 1.5 meters per second, such as about 1 meter per second or less, such as about 1 meter per second, such as about 0.9 meters per second, such as about 0.5 meters per second.

In some embodiments, the refrigerator appliance may also include a camera. For example, the camera may be positioned within the refrigerator appliance whereby the aging chamber is within a field of view of the camera, such as when the aging chamber door is in the closed position. In particular, at least a portion of the shelves 150 in the aging chamber may be within the field of view of the camera, such that contents, e.g., meat 1000, disposed on the shelves may be viewed with the camera. In some embodiments, the camera may be positioned inside the cabinet, such that the aging chamber is within the field of view of the camera regardless of the position of the aging chamber door, whereas in other embodiments, the camera may also or instead be positioned on the aging chamber door, such that the aging chamber is within the field of view of the camera at least when the aging chamber door is in the closed position. As discussed above, locking the aging chamber door during the aging time may advantageously help maintain consistent environmental conditions (e.g., temperature and humidity) within the aging chamber during the aging time. Keeping the door locked, however, impedes the user's ability to monitor the progress of the aging process. Thus, providing the camera allows the user to remotely monitor the meat aging process without disturbing the aging chamber and/or contents thereof during the aging time.

The aging process may take place over an aging time. For example, as mentioned above, the method 400 may include locking the aging chamber door at the beginning of the aging time, activating the humidity control system during the aging time, and unlocking the door at the end of the aging time. Further, the method 400 may include sending one or more user notifications (to a display on the refrigerator appliance, such as on control panel 140 or on a remote user interface device such as display 502) at certain points in the aging time. The aging time may be determined based on one or more of several factors. For example, the aging time may be determined based on a meat type, such as in response to a meat type input or meat type selection, e.g., one of the exemplary meat types depicted in FIG. 4. The aging time may also or instead be determined based on one or both of the interior volume of the aging chamber and/or the weight of the meat. For example, the volume of the aging chamber is a fixed quantity and may be stored in a memory, such as a memory of controller 144 described above, or may be accessed from a remote database, such as over the internet and/or in the cloud, where the aging chamber volume may be accessed by looking up a model number or other identifying information of the refrigerator appliance to retrieve an associated aging chamber volume. Also by way of example, the weight of the meat to be aged may be manually entered by the user, such as from a user interface on the refrigerator appliance and/or via the remote user interface device, such as in a mobile app running on a smartphone or tablet, etc. Thus, in some embodiments, the aging time, e.g., in days, may be calculated based on the volume of the aging chamber and the weight or volume of the meat to be aged, as well as a meat type, where the meat type may correspond to a cut of meat.

In some embodiments, exemplary methods may include and/or exemplary refrigerator appliances may be configured for performing a clean cycle after the aging process is complete and the aged meat has been removed from the aging chamber. For example, in some embodiments, the end of the aging process may include unlocking the door, such as by moving the lock to an unlocked position after the aging time has elapsed. The removal of the aged meat may then be detected by, e.g., detecting a door opening of the aging chamber door after moving the lock to the unlocked position. A subsequent door closing of the aging chamber door may also be detected after the door open and before initiating the clean cycle. After detecting the door opening of the aging chamber door, the clean cycle maybe initiated. The clean cycle may include activating the humidity control system throughout the entire clean cycle, such as activating both the fan and the UV light continuously throughout the clean cycle. The clean cycle may also include locking the aging chamber door at the beginning of the clean cycle. The clean cycle may have a duration between about ten minutes and about forty minutes, such as between about fifteen minutes and about thirty minutes, such as about twenty minutes or about thirty minutes. When the clean cycle is complete, the door may be unlocked again, and the aging chamber 123 may thus be ready for further use, such as an additional meat aging operation, or a convertible chamber operation as mentioned above.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A refrigerator appliance comprising:
a cabinet;
a fresh food chamber defined in the cabinet;
a freezer chamber defined in the cabinet;
an aging chamber defined in the cabinet;
a dedicated humidity control system in fluid communication with the aging chamber and fluidly isolated from the fresh food chamber and the freezer chamber;
an aging chamber door mounted to the cabinet whereby the aging chamber door is movable relative to the cabinet between a closed position where the aging chamber is sealingly enclosed by the aging chamber door and an open position permitting access to the aging chamber;
a lock configured to retain the aging chamber door in the closed position; and
a controller, the controller configured for:
receiving a meat aging input from a user interface of the refrigerator appliance;
moving the lock to a locked position whereby the aging chamber door is retained in the closed position in response to the meat aging input; and
activating the dedicated humidity control system for an aging time after moving the lock to the locked position.

2. The refrigerator appliance of claim 1, wherein the dedicated humidity control system comprises a light configured to emit ultraviolet light.

3. The refrigerator appliance of claim 2, wherein activating the dedicated humidity control system comprises activating the light periodically throughout the aging time, whereby ultraviolet light is directed into the aging chamber while the light is activated.

4. The refrigerator appliance of claim 1, wherein the dedicated humidity control system comprises a fan.

5. The refrigerator appliance of claim 4, wherein activating the dedicated humidity control system comprises continuously rotating the fan at a constant speed throughout the aging time, whereby air is motivated to circulate within the aging chamber.

6. The refrigerator appliance of claim 1, further comprising a camera positioned within the refrigerator appliance whereby the aging chamber is within a field of view of the camera when the aging chamber door is in the closed position, wherein the controller is further configured for receiving an image from the camera and transmitting the image to a display.

7. The refrigerator appliance of claim 1, further comprising a meat aging input selector on the refrigerator appliance, wherein the controller is configured for receiving a meat aging input from the meat aging input selector.

8. The refrigerator appliance of claim 1, wherein the controller is further configured for receiving a meat type input and determining the aging time based on the meat type input.

9. The refrigerator appliance of claim 1, wherein the controller is further configured for setting a temperature in the aging chamber to an aging temperature in response to the meat aging input.

10. The refrigerator appliance of claim 1, wherein the controller is further configured for moving the lock to an unlocked position after the aging time has elapsed, detecting a door opening of the aging chamber door after moving the lock to the unlocked position, and initiating a clean cycle after detecting the door opening of the aging chamber door.

11. A method of operating a refrigerator appliance, the refrigerator appliance comprising a cabinet with a fresh food chamber, a freezer chamber, and an aging chamber each defined in the cabinet, the refrigerator appliance further comprising an aging chamber door mounted to the cabinet whereby the aging chamber door is movable relative to the cabinet between a closed position where the aging chamber is sealingly enclosed by the aging chamber door and an open position permitting access to the aging chamber, the method comprising:
receiving a meat aging input from a user interface of the refrigerator appliance;
moving a lock configured to retain the aging chamber door in the closed position to a locked position whereby the aging chamber door is retained in the closed position in response to the meat aging input; and
activating a dedicated humidity control system in fluid communication with the aging chamber and fluidly isolated from the fresh food chamber and the freezer chamber for an aging time after moving the lock to the locked position.

12. The method of claim 11, wherein the dedicated humidity control system comprises a light configured to emit ultraviolet light.

13. The method of claim 12, wherein activating the dedicated humidity control system comprises activating the light periodically throughout the aging time, whereby ultraviolet light is directed into the aging chamber while the light is activated.

14. The method of claim 11, wherein the dedicated humidity control system comprises a fan.

15. The method of claim 14, wherein activating the dedicated humidity control system comprises continuously rotating the fan at a constant speed throughout the aging time, whereby air is motivated to circulate within the aging chamber.

16. The method of claim 11, wherein the refrigerator appliance further comprises a camera positioned within the refrigerator appliance whereby the aging chamber is within a field of view of the camera when the aging chamber door is in the closed position, the method further comprising receiving an image from the camera and transmitting the image to a display.

17. The method of claim 11, wherein receiving the meat aging input from the user interface of the refrigerator appliance comprises receiving the meat aging input from a user interface of a remote user interface device in wireless communication with the refrigerator appliance.

18. The method of claim 11, further comprising receiving a meat type input and determining the aging time based on the meat type input.

19. The method of claim 11, further comprising setting a temperature in the aging chamber to an aging temperature in response to the meat aging input.

20. The method of claim 11, further comprising moving the lock to an unlocked position after the aging time has elapsed, detecting a door opening of the aging chamber door after moving the lock to the unlocked position, and initiating a clean cycle after detecting the door opening of the aging chamber door.
